(19) United States
(12) Patent Application Publication
(10) Pub. No.: US 2026/0155203 A1
(43) Pub. Date: Jun. 4, 2026

(54) DEVICE AND METHOD FOR PREDICTING RISK OF DISEASE INCIDENCE

(71) Applicant: GENOPLAN INC., New York, NY (US)

(72) Inventors: Byung Kyu Kang, Camden, DE (US); Byung Chul Lee, Seoul (KR); Ji Woong Kim, Seoul (KR); Young Kee Lee, Seoul (KR); Myeong Jae Cheon, Seoul (KR); Joon Ho Kang, Seoul (KR)

(21) Appl. No.: 19/122,304

(22) PCT Filed: Oct. 18, 2023
(86) PCT No.: PCT/KR2023/016185

§ 371 Date: Apr. 18, 2025

(30) Foreign Application Priority Data
Oct. 18, 2022 (KR) 10-2022-0133674
Oct. 18, 2022 (KR) 10-2022-0133676

(51) Int. Cl. G16B 20/20; G16B 40/30

(52) U.S. Cl. CPC G16B 20/20; G16B 40/30

(57) ABSTRACT

The present disclosure relates to a device and method for predicting the risk of disease occurrence by utilizing single nucleotide polymorphisms and the presence or absence of monogenic variants in a subject. According to the device and method according to an aspect, prediction of the risk of disease occurrence is enabled based on a more accurate genetic risk by integrating together monogenic variants, which are based on the subject's genetic information and have a clear causal relationship but appear rarely, and the polygenic risk score, which is based on commonly occurring single nucleotide polymorphisms that, in comparison, do not have high individual association.

### TECHNICAL FIELD

The present disclosure relates to a device and method for predicting disease occurrence risk based on a subject's single nucleotide polymorphisms and the presence or absence of monogenic variants.

### BACKGROUND ART

A single nucleotide polymorphism (SNP) is a type of genetic variation exhibiting differences in genetic base sequences between individuals, representing a position where a single nucleotide differs, and where bi-allelic variation occurs with a frequency of 1% or more within a population.

Recently, with the development of genomic analysis technologies such as genome-wide association studies (GWAS) and next-generation sequencing (NGS), technologies capable of analyzing human genomic variation, especially SNP information, have been developed.

In recent studies, while individual SNPs generally exhibit low disease association, it is being revealed that specific combinations of SNPs can exhibit high disease association. To discover optimal SNP combinations capable of predicting disease occurrence, methods such as Bayes factor, logistic regression analysis, hidden Markov model, support vector machine, and random forest machine learning are being used in recent studies.

GWAS analysis is an exploratory method for finding traits associated with genetic variation (e.g., height, hair color, eye color, various disease risks), and generally employs a method wherein genetic information across the entire genome region is compared between cases (Case, a group having the trait of interest, e.g., a patient group) and controls (Control, a group not having the trait, e.g., a normal group), and genetic variants having a higher frequency in the case group are selected as genetic variants associated with the trait.

Accordingly, the present disclosure was completed by constructing a model that predicts disease occurrence risk by incorporating both numerous genetic variants identified through GWAS analysis and the presence or absence of monogenic factor variations in specific genes known to cause the disease.

### DISCLOSURE OF INVENTION

### Technical Problem

One aspect provides a method of predicting disease risk based on the genetic risk of disease, the method including: selecting single nucleotide polymorphisms (SNPs) associated with disease occurrence and a monogenic variant associated with disease occurrence; analyzing, using a sample from a subject, the subject's information on the selected SNP and the subject's information on the selected monogenic variant; and obtaining a first value, derived from the selected SNP information and weighted in proportion to the SNP's influence on disease occurrence, and a second value, derived from the selected monogenic variant information and weighted in proportion to the monogenic variant's influence on disease occurrence.

Another aspect provides a computer-readable recording medium having recorded thereon a program for executing the aforementioned method on a computer.

Another aspect provides a computing device including at least one memory and at least one processor, wherein the processor is configured to: select single nucleotide polymorphisms (SNPs) associated with disease occurrence and a monogenic variant associated with disease occurrence; analyze, using a sample from a subject, the subject's information on the selected SNP and the subject's information on the selected monogenic variant; and obtain a first value, derived from the selected SNP information and weighted in proportion to the SNP's influence on disease occurrence, and a second value, derived from the selected monogenic variant information and weighted in proportion to the monogenic variant's influence on disease occurrence, thereby predicting the risk of a disease based on the genetic risk of the disease.

### Solution to Problem

The present disclosure provides a method of predicting a subject's risk of developing a specific disease, the method including: selecting gene groups or genes associated with the specific disease by using, as an indicator for any gene, the odds ratio (OR) for the specific disease, the population attributable fraction (PAF), or the product of the OR and PAF; and analyzing genetic variations of the subject within the selected gene groups or genes. Specifically, the selection of gene groups or genes associated with the occurrence of a specific disease may include clustering genes having a similar degree of association or influence on the occurrence of that specific disease. More specifically, the OR and PAF for a specific disease, either individually or their product, may be used as an index for selecting genes that carry monogenic variants related to the specific disease. This proceeds, in selecting disease-associated genes, not by a theoretical approach but by a method of estimating from actual clinical data, specifically from genomic data of disease groups and non-disease groups.

The disease may include, without limitation, all diseases caused by genetic factors or upon which the influence of genetic factors acts directly or indirectly, and specifically may be ovarian cancer, stomach cancer, breast cancer, prostate cancer, cardiovascular disease, metabolic disease, or diabetes, without being limited thereto. In an embodiment, the disease may be breast cancer or prostate cancer.

According to the present disclosure, the method of predicting a subject's risk of developing a specific disease based on the genetic risk of disease may improve the accuracy of disease risk prediction by newly discovering and incorporating variation information not only from genes whose correlation with the target disease is already well-known, but also from genes that are potentially highly relevant but not yet well-established.

In the present specification, the term “gene” refers to a fragment of a nucleic acid sequence that encodes a protein or RNA (also referred to herein as a “coding sequence” or “coding region”), which may, in certain cases, include regulatory regions located upstream or downstream of the coding sequence, such as promoters, operators, or terminators.

In the present specification, the term “genetic information” encompasses information obtained through gene analysis of a subject, including, for example, information regarding genetic traits or gene variations related to specific disease occurrence. The genetic variation may be in the form of missense variants, frameshift mutations, nonsense mutations, splice mutations, substitutions, insertions, or deletions of nucleotides, without being limited thereto. In a particular example, the genetic information may include single nucleotide polymorphisms (SNP). The disease occurrence risk calculated based on such genetic information includes the congenital occurrence risk for the corresponding disease.

In the present specification, “polymorphism” refers to a case where two or more alleles exist at a single gene locus, and among polymorphic sites, one where only a single base differs between individuals is called a single nucleotide polymorphism (SNP). A preferred polymorphic marker has two or more alleles exhibiting an occurrence frequency of 1% or more, more preferably 5% or 10% or more, in a selected population.

In the present specification, “odds ratio (OR)” serves as an index estimating relative risk and is typically estimated from study data (e.g., cohort or case-control studies). OR is calculated by dividing the ratio of patients (cases) to controls among individuals carrying a rare variant in a specific gene by the ratio of patients (cases) to controls among individuals not carrying the variant. This is considered the odds ratio for the gene.

In the present specification, “population attributable fraction (PAF)” indicates a numerical inference of the impact in cases where it is estimated that a specific disease occurred due to exposure to a specific external factor, and is defined by Equation 1 below. For example, in a situation where lung cancer is estimated to have occurred due to exposure to the external factor of smoking, it indicates the numerically inferred influence of smoking on lung cancer occurrence. In the context of the present disclosure, PAF may indicate the numerically inferred influence of genetic factors, such as variations in specific genes, on the occurrence of a specific disease.

$$PAF = \frac{{p}_{e} ( RR - 1 )}{{p}_{e} ( RR - 1 ) + 1} \qquad [ Equation 1 ]$$

The term pe represents the proportion exposed to the risk factor (prevalence of risk factors), and RR (relative risk) represents the ratio of the outcome probability in the group exposed to the risk factor to the outcome probability in the group not exposed. For example, in the situation where lung cancer is estimated to have occurred due to exposure to the external factor of smoking, and when PAF represents the numerically inferred influence of smoking on lung cancer occurrence, then Pe in the equation for calculating PAF indicates the proportion exposed to smoking. In the context of the present disclosure, when PAF represents the numerically inferred influence of genetic factors, such as variations in genes, on the occurrence of a specific disease, pe may indicate the proportion carrying the genetic variation in the prediction of disease occurrence risk.

The present disclosure relates to a device for predicting a subject's genetic risk of disease occurrence, and the device for predicting the risk of disease occurrence may include analyzing a sample from a subject to detect the presence or absence of variations in a monogenic variant associated with disease occurrence.

One aspect provides a method of predicting the risk of disease based on the genetic risk of disease, the method including: selecting single nucleotide polymorphisms (SNPs) associated with disease occurrence and a monogenic variant associated with disease occurrence; analyzing, using a sample from a subject, the subject's information on the selected SNP and the subject's information on the selected monogenic variant; and obtaining a first value, derived from the selected SNP information and weighted in proportion to the SNP's influence on disease occurrence, and a second value, derived from the selected monogenic variant information and weighted in proportion to the monogenic variant's influence on disease occurrence.

In an embodiment, the first value may be a polygenic risk score (PRS) value obtained based on the subject's single nucleotide polymorphism information for the disease.

In an embodiment, the second value may be a monogenic risk score (MRS) value obtained based on the subject's monogenic factor information for the disease.

Another aspect includes a recording medium having recorded thereon a method of executing the above-described method on a computer.

Another aspect provides a computing device, including at least one memory and at least one processor, wherein the processor is configured to: select single nucleotide polymorphisms (SNPs) associated with disease occurrence and a monogenic variant associated with disease occurrence; analyze, using a sample from a subject, the subject's information on the selected SNP and the subject's information on the selected monogenic variant; and obtain a first value, derived from the selected SNP information and weighted in proportion to the SNP's influence on disease occurrence, and a second value, derived from the selected monogenic variant information and weighted in proportion to the monogenic variant's influence on disease occurrence, to thereby predict the risk of the disease based on the genetic risk of the disease.

In an embodiment, calculation of an integrated genetic risk for the disease based on the first value and the second value thus obtained may be further included.

The analysis of the subject's genetic information includes performing a procedure that involves a physical change in a biological sample isolated from the subject, specifically blood, tissue, or cell samples, for example, a biopsy or isolated nucleic acid (such as DNA or RNA) sample.

Such a physical change may include cutting or fragmenting a physical material, for instance producing a physically isolated entity from a fragment of genomic DNA (for example, isolating a nucleic acid sample from tissue), combining two or more separate entities into a mixture, or performing a chemical reaction that destroys or forms covalent or non-covalent bonds.

In an embodiment, the sample from the subject may be blood, wherein the blood may preferably be whole blood, serum, plasma, or peripheral blood mononuclear cells, but is not limited thereto.

The method of predicting disease risk may include calculating a polygenic risk score (PRS) and a monogenic risk score (MRS) by using genetic variants contained in a sample separated from the subject.

In an embodiment, the first value may indicate the polygenic risk score (PRS) of a biological sample isolated from the subject, as used in the device for predicting the risk of disease occurrence.

In an embodiment, the second value may indicate the monogenic risk score (MRS) of a biological sample isolated from the subject, as used in the device for predicting the risk of disease occurrence.

In an embodiment, a “monogenic variant” may be used interchangeably with a “pathogenic variant,” referring to a genetic variation that can act as a cause of a particular disease. Consequently, individuals carrying such a monogenic variant (or pathogenic variant) may be at a several-fold increased risk for that particular disease. However, the number of genetic variants an individual can carry at a specific locus is 0, 1, or 2, and in the case of the pathogenic variants, it is reported that over 99% of individuals carry no variant at all. As a result, the frequency of carrying a given single variant is below 1%, which is extremely rare. Therefore, while monogenic variants can be highly advantageous for predicting disease occurrence risk, they also exhibit the limitation that its frequency is extremely rare.

In an embodiment, the monogenic variant may include, not only genes that directly cause the disease, but also other genes that, from a genomics perspective, could influence the causative gene.

In an embodiment wherein the disease is breast cancer, the gene(s) acting as a monogenic variant for breast cancer may include one or more selected from the group consisting of BRCA1, BRCA2, PALB2, ATM, CDH1, CHEK2, BARD1, TP53, MYTYH, NF1, RAD51C, BRIP1, and RAD51D.

In an embodiment wherein the disease is prostate cancer, the gene(s) acting as a monogenic variant for prostate cancer may include one or more selected from the group consisting of HOXB13, ATM, BRCA2, PTEN, CDH1, PMS2, CHEK2, BRCA1, MSH6, MSH2, BARD1, PALB2, TP53, and NBN.

In an embodiment, the type(s) of gene(s) acting as a cause for the disease occurrence may vary based on information regarding the subject's age, sex, race, etc., and the dataset for the machine learning may include genetic information from individuals diagnosed with the disease and genetic information from individuals not diagnosed with the disease.

In an embodiment, the machine learning may involve reflecting the influence of genetic variations on the development of the disease as an effect size.

In the method of the present disclosure for providing information to predict disease occurrence risk, the monogenic variant associated with disease occurrence may be selected based on an odds ratio (OR) for the probability of disease occurrence, a population attributable fraction (PAF), or a product of OR and PAF for the probability of disease occurrence.

In an embodiment, utilizing the OR and PAF regarding the probability of occurrence for a specific disease, machine learning for the selection and clustering of genes highly relevant to the disease may be performed.

In an embodiment, the consideration of weights in proportion to the influence on disease occurrence, with respect to the second value, may include clustering one or more monogenic variants into one or more clusters.

Such clustering may utilize any one unsupervised learning technique selected from the group consisting of hierarchical clustering, k-means clustering, mixture model clustering, density-based spatial clustering of applications with noise (DBSCAN), generative adversarial networks (GAN), and self-organizing map (SOM), but is not limited thereto.

In an embodiment of the present disclosure, by applying the density-based clustering method (DBSCAN) to the log-transformed values of the OR and PAF regarding the probability of specific disease occurrence, clustering may be performed by genes having similar influence on specific disease occurrence. Here, the relevance or influence of each cluster on disease occurrence may exhibit specific patterns. In an embodiment, by the density-based clustering method, each resulting cluster, particularly as its distance from the origin increases, may be selected as genes having higher relevance to disease occurrence, but is not limited thereto.

In an embodiment, the selection and clustering of monogenic variants related to disease occurrence may be performed by using the product of the odds ratio (OR) for the probability of disease occurrence and the population attributable fraction (PAF). By sorting the product values in descending order, genes ranked higher in the list may be selected as genes having higher relevance to the disease occurrence.

In an embodiment, when selecting the monogenic variants related to the disease occurrence, genes among the genetic variants included in the dataset that have a frequency of less than 0.001% may be excluded.

In an embodiment, the second value may be determined by the presence or absence of a genetic variation in a monogenic variant selected as being associated with disease occurrence. In the present embodiment, after selecting monogenic variants from a genomic dataset, effect sizes for each gene were estimated using actual incidence data, and the second value was determined by assigning weights according to those effect sizes.

In an embodiment, wherein the disease is breast cancer, the second value may be determined by the presence or absence of one or more types of genetic variant selected from the group consisting of BRCA1, BRCA2, ATM, PALB2, CHEK2, BARD1, RAD51C, MUTYH, BRIP1, RAD51D, CHD1, TP53, SDHB, and NF1.

In an embodiment, wherein the disease is prostate cancer, the second value may be determined by the presence or absence of one or more types of genetic variant selected from the group consisting of HOXB13, ATM, BRCA2, PTEN, CDH1, PMS2, CHEK2, BRCA1, MSH6, MSH2, BARD1, PALB2, TP53, and NBN.

In an embodiment, the polygenic risk score (PRS) may be a method, derived through genome-wide association studies (GWAS), for confirming association with the development of a specific disease, even if the variants involved do not act as a direct cause thereof. The PRS is a method of measuring the risk of a specific disease caused by congenital or inherited factors, and its influence may be increased when multiple genetic factors are incorporated into a prediction model. Specifically, the PRS may refer to a value that has undergone a process of modulating the influence values of genetic variations to reflect the characteristics of a specific disease, such as by assigning weights to single nucleotide polymorphisms (SNPs) or specific SNPs and undergoing a numerical quantification process.

In an embodiment, the first value may be determined by whether the subject carries an SNP genetic variant whose disease association corresponds to the top 10th percentile or that occurs with a frequency at least twice that observed in the control group.

In an embodiment, the SNP variant may be an insertion or deletion of 50 or fewer base pairs.

In an embodiment, wherein the disease is breast cancer, a determination related to specific SNPs identified for predicting breast cancer risk may be based on the presence or absence of one or more variations selected from the group consisting of rs11200014, rs78540526, rs4784227, rs4442975, rs62355901, and rs10941679.

In an embodiment, wherein the disease is prostate cancer, a determination related to specific SNPs identified for predicting prostate cancer risk may be based on the presence or absence of one or more variations selected from the group consisting of rs10090154, rs11263763, rs56005245, rs12795301, rs191785584, and rs6998061.

In an embodiment, the type(s) of specific SNPs acting as a cause for the disease occurrence may vary based on information about the subject's age, sex, race, and the like.

In an embodiment, the PRS and MRS may each be calculated by taking into account the effect size for the SNP analysis and for the selected monogenic variants.

In an embodiment, the effect size may be weighted according to whether the factors are in a group with high disease association or in a group with lower disease association, in descending order of association strength.

In an embodiment, the first value and second value may be calculated by assigning weights in proportion to the effect size of each genetic variant.

In an embodiment, classification of the subject into a non-risk group, a risk group, a high-risk group, or a very high-risk group according to the risk of disease occurrence may be further included.

The prediction of disease occurrence risk may include searching for disease-associated genetic variants. Specifically, the prediction of disease occurrence risk may refer to calculating, based on the results of analyzing the subject's sample, the subject's polygenic risk score (PRS) via a predetermined machine learning model (or algorithm) upon inputting the subject's single nucleotide polymorphism (SNP) genetic variation data; calculating the subject's monogenic risk score (MRS) via a predetermined machine learning model (or algorithm) upon inputting the subject's monogenic factor genetic variation data; or summing the calculated PRS and MRS.

In an embodiment, the processor in the device for predicting the risk of disease occurrence may, using a machine learning model, analyze genetic information including the subject's single nucleotide polymorphism (SNP) information to calculate a first value, analyze genetic information including the subject's monogenic variant information to calculate a second value, and calculate the risk of disease occurrence using the first value and the second value. In this case, the machine learning model may be trained to: select a single nucleotide polymorphisms (SNP) associated with disease occurrence and a monogenic variant associated with disease occurrence; analyze, using a sample from the subject, the subject's information on the selected SNP and the subject's information on the selected monogenic variant; calculate, respectively, a first value, derived from the selected SNP information and weighted in proportion to the SNP's influence on disease occurrence, and a second value, derived from the selected monogenic variant information and weighted in proportion to the monogenic variant's influence on disease occurrence; and classify the subject into a non-risk group, a risk group, a high-risk group, or a very high-risk group according to the calculated disease occurrence risk.

In an embodiment, the prediction of disease occurrence risk may involve using an artificial intelligence model to train a weighted risk model for determining disease occurrence risk.

This weighted risk model may be formed by summing the number of risk alleles for the disease-associated SNPs and monogenic variants in each subject, while assigning weights according to the effect size (contribution) of each SNP or monogenic variant to the disease. Each subject's SNP and monogenic variant risk alleles may be present in 0, 1, or 2 copies.

In an embodiment, data set for the machine learning may include the genetic information of individuals diagnosed with the disease and the genetic information of individuals not diagnosed with the disease.

In an embodiment, the machine learning may involve reflecting the effect sizes of genetic variants that influence the development of the disease.

In an embodiment, the effect size may be weighted in order, beginning with a group of factors having high association with disease occurrence and progressing to a group of factors having low association.

To calculate the disease occurrence risk, algorithms and/or techniques such as a logistic regression model, a support vector machine, a decision tree, a nearest-neighbor classifier, a neural network, a random forest, or a boosted tree may be used in machine learning, without limitation.

In an embodiment, Equation 2 may be used to predict the disease occurrence risk.

$$y = F ( P ( x\_p ) , M ( x\_m ) ) \qquad [ Equation 2 ]$$

P(x_p) is a polygenic risk score (or label),
x_p is a set of SNP markers associated with disease occurrence,
M(x_m) is a monogenic risk score (or label),
x_m is a set of monogenic variants associated with disease occurrence, and
F(x) is the disease occurrence risk level or estimated onset value produced by combining the two risk scores, P and M.

In an embodiment, F(x) may be a logistic regression model or a support vector machine, without being limited thereto, and its accuracy (performance) may vary depending on the algorithm.

In an embodiment, by using a support vector machine in machine learning, it is possible to classify the subject into a non-risk group, a risk group, a high-risk group, or a very high-risk group according to the calculated disease occurrence risk.

However, the aforementioned algorithms and/or methods are only illustrative and are not intended to limit the spirit of the present disclosure.

### Advantageous Effects of Invention

According to one aspect, the device and method for predicting the risk of disease occurrence in a subject can predict the risk of disease occurrence by integrating both monogenic factors, which are based on the subject's genetic information and have a clear causal relationship but appear rarely, and the polygenic risk score, which is based on commonly occurring single nucleotide polymorphisms that, in comparison, do not have high individual association, thereby calculating a more accurate genetic risk.

### BRIEF DESCRIPTION OF THE DRAWINGS

FIG. 1 is a flowchart illustrating an example of a method of predicting disease occurrence risk according to an embodiment.

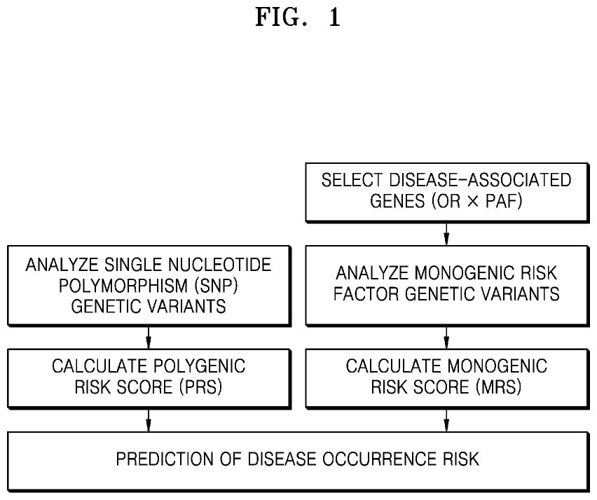

FIG. 2 is a flowchart illustrating another example of a method of predicting disease occurrence risk according to an embodiment.

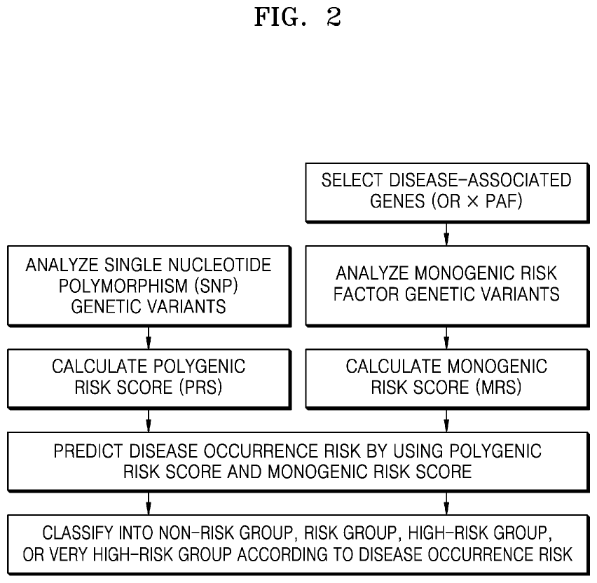

FIG. 3 is a flowchart illustrating one example of calculating a polygenic risk score according to an embodiment.

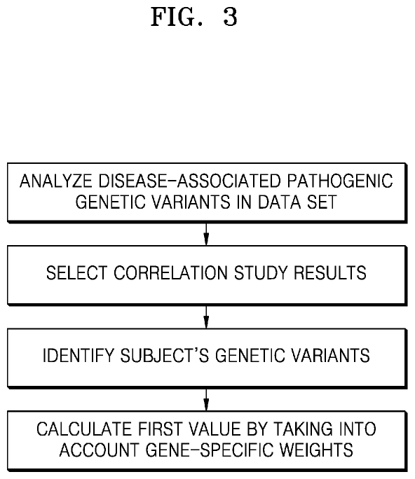

FIG. 4 is a flowchart illustrating one example of calculating a monogenic risk score according to an embodiment.

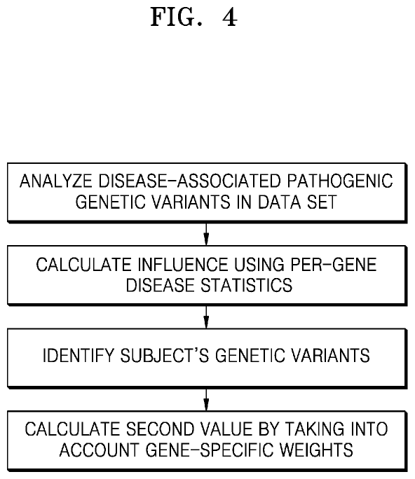

FIG. 5 is a graph illustrating an example of selecting and clustering monogenic variants for breast cancer, carried out by machine learning, according to an embodiment.

FIG. 6 is a graph showing a correlation between a polygenic risk score and each monogenic variant cluster of breast cancer, according to an embodiment.

FIG. 7 is a graph illustrating an example of a process for selecting and clustering monogenic variants for prostate cancer, carried out by machine learning, according to an embodiment.

FIG. 8 is a graph showing a correlation between a polygenic risk score and each monogenic variant cluster for prostate cancer, according to an embodiment.

### BEST MODE FOR CARRYING OUT THE INVENTION

Hereinafter, preferred embodiments will be described to facilitate understanding of the present disclosure. However, the following embodiments are provided only to facilitate easier understanding of the present disclosure and are not intended to limit the scope of the disclosure.

In an embodiment of the method of predicting the risk of disease based on the genetic risk of disease, wherein the disease is breast cancer or prostate cancer, the risk levels for breast cancer and prostate cancer are predicted based on the genetic risk associated with each respective disease.

### Example 1.1. Selection of Breast Cancer-Related Genes—Based on OR×PAF Value

Based on breast cancer diagnosis status and the presence or absence of a monogenic variant, samples were categorized as follows: (a) samples from individuals with breast cancer carrying a monogenic variant, (b) samples from individuals with breast cancer not carrying a monogenic variant, (c) samples from individuals without breast cancer carrying a monogenic variant, and (d) samples from individuals without breast cancer not carrying a monogenic variant. The respective counts were substituted into the following equations to calculate gene-specific statistical metrics.

First, the p-value was calculated using Fisher's exact test, and subsequently, odds ratio (OR), relative risk (RR), exposed proportion, and population attributable fraction (PAF) were calculated using the following equations. These values are presented in Table 1. Genes exhibiting an RR value of less than 2 were excluded.

$$Odds Ratio ( OR ) = \frac{\frac{\begin{matrix}Number of diseased individuals \\ with monogenic variant ( a )\end{matrix}}{\begin{matrix}Number of diseased individuals \\ without monogenic variant ( b )\end{matrix}}}{\frac{\begin{matrix}Number of non - diseased individuals \\ with monogenic variant ( c )\end{matrix}}{\begin{matrix}Number of non - diseased individuals \\ without monogenic variant ( d )\end{matrix}}} \qquad [ Equation 3 ]$$

$$Relative Risk ( RR ) = \frac{\frac{\begin{matrix}Number of diseased individuals \\ with monogenic variant ( a )\end{matrix}}{\begin{matrix}Total number of diseased \\ individuals ( a + b )\end{matrix}}}{\frac{\begin{matrix}Number of non - diseased individuals \\ with monogenic variant ( c )\end{matrix}}{\begin{matrix}Total number of non - diseased \\ individuals ( c + d )\end{matrix}}} \qquad [ Equation 4 ]$$

$$exposed proportion = \frac{\begin{matrix}Total number of diseased \\ individuals ( a + b )\end{matrix}}{\begin{matrix}Total number of diseased and \\ non - diseased individuals ( a + b + c + d )\end{matrix}} \qquad [ Equation 5 ]$$

TABLE 1

| | | | | | | exposed | |
|---|---|---|---|---|---|---|---|
| o | gene | OR*PAF | OR | p-value | RR | proportion | PAF |
| 1 | BRCA1 | 0.09254235 | 14.06872 | 2.E−20 | 14.03062 | 0.000508 | 0.006578 |
| 2 | BRCA2 | 0.07326647 | 6.87475 | 2.E−35 | 6.82972 | 0.001848 | 0.010657 |
| 3 | ATM | 0.02555615 | 4.11091 | 4.E−19 | 4.09173 | 0.002023 | 0.006217 |
| 4 | PALB2 | 0.02114053 | 4.28661 | 1.E−15 | 4.27100 | 0.001515 | 0.004932 |
| 5 | CHEK2 | 0.00840107 | 2.73503 | 2.E−08 | 2.72795 | 0.001783 | 0.003072 |
| 6 | BARD1 | 0.00406168 | 3.54031 | 2.E−04 | 3.53713 | 0.000453 | 0.001147 |
| 7 | RAD51C | 0.00365523 | 4.51247 | 1.E−03 | 4.50984 | 0.000231 | 0.000810 |
| 8 | MUTYH | 0.00335833 | 1.17757 | 2.E−02 | 1.17421 | 0.016417 | 0.002852 |
| 9 | BRIP1 | 0.00285680 | 2.79108 | 9.E−04 | 2.78869 | 0.000573 | 0.001024 |
| 10 | RAD51D | 0.00106846 | 3.08452 | 4.E−02 | 3.08365 | 0.000166 | 0.000346 |
| 11 | CDH1 | 0.00103956 | 8.01808 | 2.E−01 | 8.01750 | 0.000018 | 0.000130 |
| 12 | TP53 | 0.00100285 | 4.00950 | 7.E−02 | 4.00875 | 0.000083 | 0.000250 |
| 13 | SDHB | 0.00100285 | 4.00950 | 7.E−02 | 4.00875 | 0.000083 | 0.000250 |
| 14 | NF1 | 0.00066858 | 4.00925 | 1.E−01 | 4.00875 | 0.000055 | 0.000167 |

As shown in Table 1, when the product of OR and PAF for each gene was listed in descending order, it was confirmed that the top-ranked genes corresponded with the 9 genes (ATM, BRCA1, BRCA2, CHEK2, PALB2, BARD1, RAD51C, RAD51D, TP53) reported in the reference L. Dorling et al., N Engl J Med 2021; 384:428-439, to be associated with breast cancer risk due to protein-truncating variants.

Based on this result, the product of OR and PAF values for each gene can be utilized as a statistical genetics indicator for selecting genes related to a specific disease. This approach will be applicable not only to the selection of breast cancer-related genes as in this embodiment but also to other diseases caused by genetic factors. Gene selection using the aforementioned method is significant in that it can identify not only genes already well-known in relation to the target disease but also potentially highly relevant genes that are not yet well-established.

The breast cancer-related genes selected via the aforementioned method are incorporated as monogenic variants for predicting the development of breast cancer according to the present disclosure.

### Example 1.2. Calculation of SNP-Based Polygenic Risk Score

Female data was selected as the target dataset for modeling, and a dataset was obtained containing genomic data QC and information on breast cancer diagnosis status for 13,581 women diagnosed with breast cancer and 117,248 women not diagnosed with breast cancer (Control). Among GWAS results for breast cancer, after evaluating factors such as ethnicity, sample size, and methodology, Nature 551, 92-94 (2017) was selected, and ‘Pruning and Thresholding’, one of the approaches for calculating polygenic risk scores, was applied using the marker set information provided in this paper. In this process, markers with low variant frequency and low quality were excluded according to general QC criteria, and as a result of applying this methodology, only markers satisfying p-value<0.0003 were included in the polygenic risk score calculation. The entire dataset was sorted according to the predicted disease risk values, and individuals were subsequently grouped and ranked from highest to lowest risk. To evaluate the PRS modeling results, comparisons were made based on metrics such as the odds ratios for breast cancer. After dividing the entire sample into 100 groups and analyzing them in clusters of 10, it was confirmed that the risk level of the top two groups was more than twice as high as that of the middle groups. This result is consistent with existing breast cancer research. However, unlike conventional methodologies (which simply compare high-risk groups with average or low-risk groups), the method classified the entire sample into 5 groups according to risk level.

The SNP-based polygenic risk score calculated through the method of Example 1.2 may be utilized in predicting breast cancer development in the present disclosure. In this process, differential weights are applied to the previously classified five groups, starting from the group with the highest probability of breast cancer development, and these weighted values are incorporated into the calculation for predicting the risk of breast cancer occurrence.

### Example 1.3. Calculation of Monogenic Risk Score (MRS)

To select monogenic variants for breast cancer, genetic variants present in female samples were collected, and functional analysis (annotation) was performed. Variants with a frequency of 5% or higher were excluded, and the pathogenicity of genetic variants was predicted. Pathogenicity was confirmed in about 1,200 genetic variants, representing 2.6% of the total sample. To statistically analyze the influence of each gene on breast cancer development, the PAF was calculated for each gene using the method described in Example 1.1 and Table 1. To classify genes that would receive weighting among the selected pathogenic genes, five gene groups were established based on the product of odds ratio and population attributable fraction for each gene, ranking from highest to lowest values. Scores of 10, 9, 8, 7, and 6 were assigned to groups in descending order of value. This scoring system was designed to assign appropriate weights even to genes with relatively lower influence. The significance lies merely in applying weights according to the influence of the genetic variants; the specific magnitude or interval of the numerical scores assigned as weights in the aforementioned Example is not intended to be limiting.

The group-specific weight values for monogenic variants calculated through the method in Example 1.3 were incorporated into the calculation for predicting the risk of breast cancer occurrence according to the present disclosure.

### Example 1.4. Prediction of Breast Cancer Risk

Using genetic information, the polygenic risk score (PRS) obtained from Example 1.2 and the monogenic risk score (MRS) obtained from Example 1.3 were combined, as shown in Tables 2 to 6 below, to predict the risk of breast cancer occurrence in a subject.

For each group classified based on the PRS obtained from Example 1.2 and the MRS from Example 1.3, the number of patients diagnosed with breast cancer and the number of women who were not diagnosed were identified.

The number of patients actually diagnosed with breast cancer and the number of controls not diagnosed with breast cancer in each group, sequentially from the group classified as having the highest PRS to the group classified as having the lowest PRS, are shown in Tables 2 to 6.

TABLE 2

| | | | | | | Breast |
|---|---|---|---|---|---|---|
| | | | Breast | Control | | cancer |
| Groups | PRS | MRS | cancer | group | Total | ratio |
| 6_5 | 5 | 6 | 7 | 4 | 11 | 63.636% |
| 10_5 | 5 | 10 | 32 | 23 | 55 | 58.182% |
| 8_5 | 5 | 8 | 15 | 18 | 33 | 45.455% |
| 9_5 | 5 | 9 | 37 | 48 | 85 | 43.529% |
| 7_5 | 5 | 7 | 77 | 278 | 355 | 21.690% |
| Novar_5 | 5 | 0 | 3681 | 17372 | 21053 | 17.484% |

TABLE 3

| | | | | | | Breast |
|---|---|---|---|---|---|---|
| | | | Breast | Control | | cancer |
| Groups | PRS | MRS | cancer | group | Total | ratio |
| 10_4 | 4 | 10 | 28 | 29 | 57 | 49.123% |
| 9_4 | 4 | 9 | 26 | 44 | 70 | 37.143% |
| 8_4 | 4 | 8 | 11 | 23 | 34 | 32.353% |
| 7_4 | 4 | 7 | 73 | 335 | 108 | 17.892% |
| 6_4 | 4 | 6 | 0 | 3 | 3 | 0.000% |
| Novar_4 | 4 | 0 | 2605 | 18405 | 21010 | 12.399% |

TABLE 4

| | | | | | | Breast |
|---|---|---|---|---|---|---|
| | | | Breast | Control | | cancer |
| Groups | PRS | MRS | cancer | group | Total | ratio |
| 10_3 | 3 | 10 | 15 | 20 | 35 | 42.857% |
| 9_3 | 3 | 9 | 26 | 47 | 73 | 35.616% |
| 8_3 | 3 | 8 | 11 | 27 | 38 | 28.947% |
| 6_3 | 3 | 6 | 3 | 9 | 12 | 25.000% |
| 7_3 | 3 | 7 | 50 | 338 | 388 | 12.887% |
| Novar_3 | 3 | 0 | 2144 | 18904 | 21048 | 10.186% |

TABLE 5

| | | | | | | Breast |
|---|---|---|---|---|---|---|
| | | | Breast | Control | | cancer |
| Groups | PRS | MRS | cancer | group | Total | ratio |
| 10_2 | 2 | 10 | 23 | 22 | 45 | 51.111% |
| 6_2 | 2 | 6 | 4 | 6 | 10 | 40.000% |
| 9_2 | 2 | 9 | 29 | 51 | 80 | 36.250% |
| 8_2 | 2 | 8 | 7 | 42 | 49 | 14.286% |
| 7_2 | 2 | 7 | 35 | 342 | 377 | 9.284% |
| Novar_2 | 2 | 0 | 1748 | 19276 | 21024 | 8.314% |

TABLE 6

| | | | | | | Breast |
|---|---|---|---|---|---|---|
| | | | Breast | Control | | cancer |
| Groups | PRS | MRS | cancer | group | Total | ratio |
| 10_1 | 1 | 10 | 29 | 34 | 43 | 43.032% |
| 9_1 | 1 | 9 | 12 | 61 | 73 | 16.438% |
| 8_1 | 1 | 8 | 4 | 34 | 38 | 10.526% |
| 7_1 | 1 | 7 | 32 | 349 | 381 | 8.399% |
| 6_1 | 1 | 6 | 0 | 8 | 8 | 0.000% |
| Novar_1 | 1 | 0 | 1212 | 19804 | 21016 | 5.767% |

As shown in Tables 2 to 6, it was confirmed that the breast cancer incidence rate is directly proportional to the PRS group. Furthermore, compared to groups without monogenic variants, groups with monogenic variants showed an increase in breast cancer incidence rates ranging from 30% to 900%, proportional to the weight level assigned to the group to which the specific gene belongs. Referring to Table 6, it can be seen that even in the group with the lowest PRS, when one or more monogenic variants are present, the breast cancer incidence rate exceeds the average within that group.

The cumulative breast cancer incidence rate of British women aged 45-74 years, referenced as a comparison dataset to that used in Example 1.2, was reported as 8.29% as of 2020, which was confirmed to be similar to the breast cancer incidence rate (8.314%) of polygenic risk score group 2 presented in Table 5.

### Example 2.1. Selection of Breast Cancer-Related Genes—DBSCAN Based

Using the same method as Example 1.1, genes highly associated with breast cancer occurrence were selected, and clustering was performed to group genes with similar influence on breast cancer development, utilizing the OR and PAF values for each gene shown in Table 1.

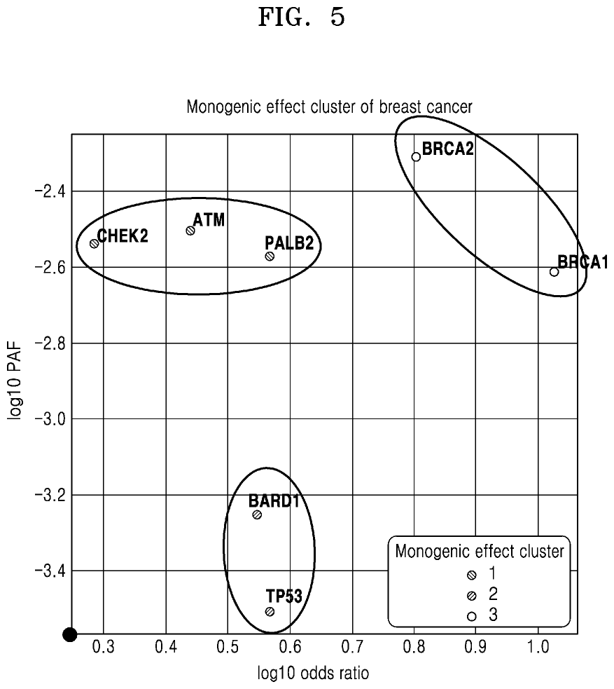

Specifically, a graph was created by plotting the logarithmic values of the OR and PAF values presented in Table 1 as the x-axis and y-axis, respectively, for each gene. Subsequently, clustering was performed among adjacent genes using DBSCAN, a density-based unsupervised clustering method, and the results are shown in FIG. 5. In this process, genes with similar influence on breast cancer development are included in a single cluster. While the influence of each cluster may exhibit specific patterns, specifically on this graph, a greater distance of the cluster from the origin indicates a greater influence on breast cancer development.

As shown in FIG. 5, among the genes associated with breast cancer development, it was confirmed that BRCA2 and BRCA1 form one cluster, CHEK2, ATM, and PALB2 form another cluster, and BARD1 and TP53 form a third cluster. Since the distances from the origin are greater in the order of BRCA2 and BRCA1>CHEK2, ATM, and PALB2>BARD1 and TP53, the magnitude of influence on breast cancer development can be expected to be proportional to this order. These results were confirmed to be similar to those from Examples 1.1 to 1.4.

### Example 2.2. Prediction of Breast Cancer Risk

The actual breast cancer risk was predicted by incorporating the SNP-based PRS from Example 1.2 into the clustering results of breast cancer-related genes and their influence as determined by the DBSCAN method in Example 2.1.

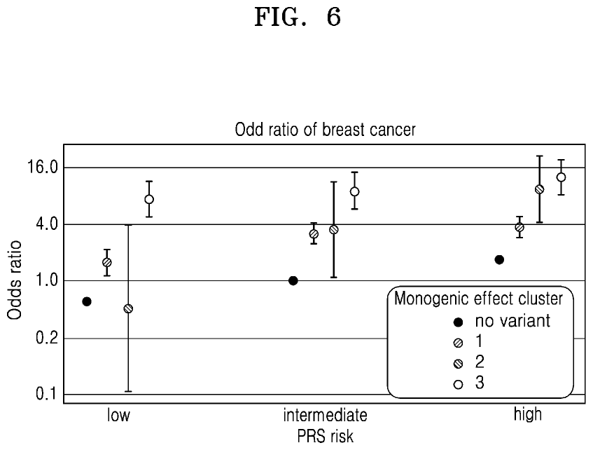

Specifically, in the same manner as in Example 1.2, groups were classified according to the PRS considering SNP variants: a low group with low genetic risk, a high group with high genetic risk, and an intermediate group in between. A graph was created with these classified groups as the x-axis and the OR for breast cancer development calculated in Table 1 as the y-axis. In this process, the case with no monogenic factor variants (no variant group) and an intermediate PRS was set as the reference with an OR value of 1.0. These results are shown in FIG. 6. Generally, an odds ratio of 1.0 indicates no association between the risk factor (monogenic variant or polymorphic variant) and the disease, while an odds ratio greater than 1.0 indicates an association between the risk factor and the disease, with higher values indicating stronger associations between the risk factor and disease development.

As shown in FIG. 6, each cluster group demonstrated odds ratios of greater magnitude in the order of high>intermediate>low PRS values. Additionally, when comparing the influence on breast cancer development between clusters, higher odds ratios were observed in the order of BRCA2 and BRCA1>CHEK2, ATM, and PALB2>BARD1 and TP53. This confirms, as observed in Example 2.1, that genes classified into clusters using DBSCAN exhibit similar influences on disease occurrence. Particularly, the cluster containing CHEK2, ATM, and PALB2 showed a wide variation in odds ratios according to PRS values, confirming that PRS has a significant influence on these genes.

These results demonstrate that in the process of predicting the risk of breast cancer occurrence through the presence or absence of breast cancer-related gene variants, more accurate prediction for the risk of breast cancer occurrence is possible when polygenic risk scores are incorporated based on the clustering of the influence of each monogenic variant using the DBSCAN method. Furthermore, this method, without being limited to breast cancer, may be usefully applied to predict the disease occurrence risk for any disease whose development is influenced by genetic variation.

### Example 3.1. Selection of Prostate Cancer-Related Genes—Using DBSCAN

Statistical criteria for each gene were determined using the same method as Example 1.1, based on prostate cancer occurrence and the presence of monogenic variants. Similarly, p-values were calculated through Fisher's exact test, and odds ratios, relative risks, exposed-proportions, and population attributable fractions (PAF) were sequentially calculated, with the values shown in Table 7. Genes with an RR value of less than 2 were excluded.

TABLE 7

| | | | | | Variant | |
|---|---|---|---|---|---|---|
| No | Gene | OR | p-value | RR | frequency | PAF |
| 1 | HOXB13 | 3.77 | 8.04E−23 | 3.08623191 | 3.54E−01 | 0.00734 |
| 2 | ATM | 2.61 | 4.46E−06 | 2.31190136 | 2.23E−01 | 0.00292 |
| 3 | BRCA2 | 2.26 | 1.59E−03 | 2.0563115 | 1.73E−01 | 0.00182 |
| 4 | PTEN | 8.59 | 2.27E−02 | 5.33627755 | 6.42E−03 | 0.00028 |
| 5 | CDH1 | 11.45 | 7.47E−02 | 6.22452011 | 1.84E−03 | 0.00010 |
| 6 | PMS2 | 0.64 | 9.22E−02 | 0.65487381 | 1.22E−01 | −0.00478 |
| 7 | CHEK2 | 1.51 | 2.10E−01 | 1.45200846 | 3.23E−01 | 0.00146 |
| 8 | BRCA1 | 0.86 | 4.85E−01 | 0.86836417 | 7.89E−01 | −0.00683 |
| 9 | MSH6 | 1.01 | 5.86E−01 | 1.00929122 | 6.79E−02 | 0.00001 |
| 10 | MSH2 | 1.01 | 5.86E−01 | 1.00929122 | 6.79E−02 | 0.00001 |
| 11 | BARD1 | 1.00 | 7.98E−01 | 0.99582676 | 2.29E−02 | −0.05844 |
| 12 | PALB2 | 1.27 | 8.66E−01 | 1.24514874 | 1.19E−01 | 0.00029 |
| 13 | TP53 | 0.88 | 8.54E−01 | 0.88911922 | 1.28E−02 | −0.00129 |
| 14 | NBN | 1.19 | 9.67E−01 | 1.17442477 | 4.86E−02 | 0.00008 |

As shown in Table 7, when listed in descending order based on the p-value for each gene, among the genes with p-values less than 0.05 showing a significant association with prostate cancer development, many genes previously known to be associated with prostate cancer risk were confirmed to be included.

The prostate cancer-related genes selected through the method in Example 3.1 are incorporated as monogenic variants in predicting prostate cancer development according to the present disclosure.

### Example 3.2. Calculation of SNP-Based Polygenic Risk Score

Male data was selected as the modeling target dataset, and a dataset was secured that included genetic data QC and information on prostate cancer diagnosis status from 8,753 men diagnosed with prostate cancer and 100,203 men not diagnosed with prostate cancer (Control). Among the GWAS results for prostate cancer, after reviewing factors such as sample size and methodology, ‘Pruning and Thresholding’, one of the approaches for calculating polygenic risk scores, was applied using marker set information. In this process, markers with low variant frequency and low quality were excluded according to general QC criteria. The entire dataset was sorted according to the predicted disease risk values, forming groups arranged from highest to lowest risk. To evaluate the PRS modeling results, comparisons were made based on metrics such as odds ratios for prostate cancer. For the purpose of assigning risk-level specific groups, the samples were classified into 3 groups.

The SNP-based polygenic risk score calculated through the method in Example 3.2 is incorporated into the prostate cancer risk prediction calculation according to the present disclosure, wherein weights are applied based on the three classified groups, starting from the group with the highest probability of prostate cancer development.

### Example 3.3. Calculation of Monogenic Risk Score (MRS)

To select monogenic variants for prostate cancer, genetic variants present in male samples were collected, and functional analysis (annotation) was performed. Variants with a frequency of 5% or higher were excluded, and only pathogenic variants among genetic variants were extracted as carrier samples. To statistically analyze the influence of each gene on prostate cancer development, OR and PAF were calculated for each gene using the method presented in Example 3.1 and Table 7. Subsequently, to select effective pathogenic genes, genes satisfying both 1) p-value<0.05, and 2) detection frequency 0.1% were selected. To select genes highly associated with prostate cancer occurrence and assign weights according to their influence on prostate cancer development, clustering was performed among similar genes using the OR and PAF values for each gene related to prostate cancer as shown in Table 7.

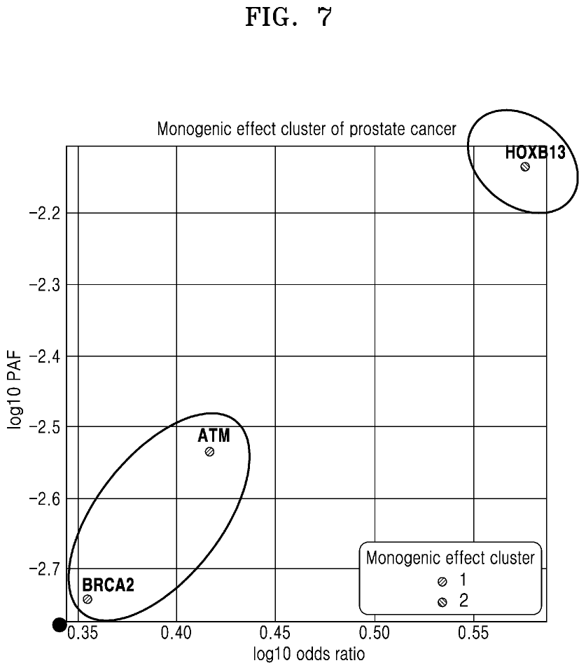

Specifically, a graph was created by plotting the logarithmic values of the OR and PAF values presented in Table 7 as the x-axis and y-axis, respectively, for each gene. Subsequently, clustering was performed among adjacent genes using DBSCAN, a density-based unsupervised clustering method, and the results are shown in FIG. 7. In this process, genes with similar influence on prostate cancer development are included in a single cluster. While the influence of each cluster may exhibit specific patterns, specifically on this graph, a greater distance of the cluster from the origin indicates a greater influence on prostate cancer development.

As shown in FIG. 7, among the genes associated with prostate cancer development, it was confirmed that HOXB13 forms one cluster, and BRCA2 and ATM form another cluster. Since the distances from the origin are greater in the order of HOXB13>BRCA2 and ATM, the magnitude of influence on prostate cancer development can be expected to be proportional to this order.

The group-specific weight values for monogenic variants calculated through the method in Example 3.3 were incorporated into the calculation for predicting the risk of prostate cancer occurrence according to the present disclosure. The significance lies merely in applying weights according to the influence of the genetic variants; the specific magnitude or interval of the numerical scores assigned as weights in the aforementioned Example is not intended to be limiting.

### Example 3.4. Prediction of Prostate Cancer Risk

The actual prostate cancer risk was predicted by incorporating the SNP-based PRS from Example 3.2 into the clustering results of prostate cancer-related genes and their influence as determined by the DBSCAN method in Example 3.3.

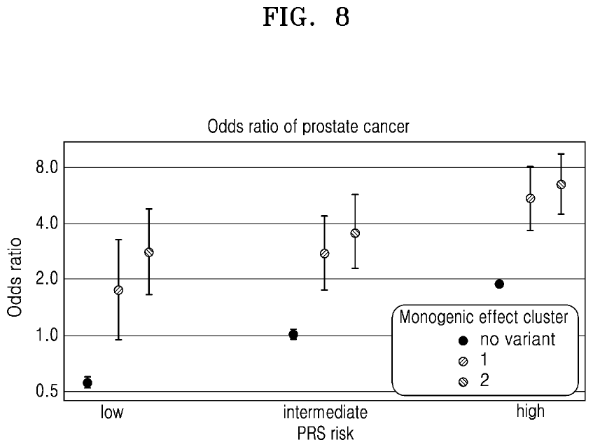

Specifically, in the same manner as in Example 3.2, groups were classified according to the PRS considering SNP variants: a low group with low genetic risk, a high group with high risk, and an intermediate group in between. A graph was created with these classified groups as the x-axis and the odds ratios for prostate cancer development calculated in Table 7 as the y-axis. In this process, the case with no monogenic factor variants (no variant group) and an intermediate PRS was set as the reference with an OR value of 1.0. These results are shown in FIG. 8. Generally, an odds ratio of 1.0 indicates no association between the risk factor (monogenic variant or polymorphic variant) and the disease, while an odds ratio greater than 1.0 indicates an association between the risk factor and the disease, with higher values indicating stronger associations between the risk factor and disease development.

As shown in FIG. 8, each cluster group demonstrated odds ratios of greater magnitude in the order of high>intermediate>low PRS values. Additionally, when comparing the influence on prostate cancer development between clusters, higher odds ratios were observed in the order of HOXB13>BRCA2, and ATM. This confirms, as observed in Example 3.3, that genes classified into clusters using DBSCAN exhibit similar influences on disease development.

These results demonstrate that in the process of predicting prostate cancer risk through the presence or absence of prostate cancer-related gene variants, more accurate prostate cancer risk prediction is possible when polygenic risk scores are incorporated based on the clustering of the influence of each monogenic variant using the DBSCAN method. Furthermore, this method, without being limited to prostate cancer, may be usefully applied to predict the disease occurrence risk for any disease whose development is influenced by genetic variation.

Using the method of the present disclosure, it was confirmed that the predicted occurrence of breast cancer or prostate cancer shows a similar trend to the actual incidence rate of breast cancer or prostate cancer within the dataset. The prediction model of the present disclosure, even without utilizing information about age or family history, can provide more accurate information about the risk of disease occurrence than approaches that consider only a single factor, such as methods that predict disease occurrence by considering only monogenic variants or methods that consider only polygenic risk scores. Specifically, even if classified into a group with relatively low polygenic risk scores or having average scores, individuals carrying monogenic variants can be classified as high-risk groups according to the gene group information. Furthermore, if an individual belongs to a group classified as having a relatively high polygenic risk score, even if no monogenic variant is present, they are classified as having a higher risk of breast cancer compared to the case where the polygenic risk score is low and no monogenic variant is present. Providing this information in advance may enable accurate prediction and prevention of disease occurrence.

1. A method of predicting a disease risk based on a genetic risk for the disease, the method comprising:
selecting a single nucleotide polymorphism (SNP) associated with disease occurrence and a monogenic variant associated with disease occurrence;
analyzing, using a sample from a subject, the subject's information on the selected SNP and the subject's information on the selected monogenic variant; and
obtaining a first value, derived from the selected SNP information and weighted in proportion to the SNP's influence on disease occurrence, and a second value, derived from the selected monogenic variant information and weighted in proportion to the monogenic variant's influence on disease occurrence.

2. The method of claim 1, further comprising calculating an integrated genetic risk for the disease based on the obtained first and second values.

3. The method of claim 1, wherein the first value is determined based on the presence or absence of an SNP genetic variant that either ranks in a top 10th percentile in terms of disease association, or occurs with a frequency at least twice that observed in a control group.

4. The method of claim 1, wherein the second value is determined based on the presence or absence of a genetic variation in the monogenic variant selected as being associated with disease occurrence.

5. The method of claim 1, wherein the selected monogenic variant associated with disease occurrence is selected based on an odds ratio (OR) related to a probability of developing the disease, a population attributable fraction (PAF), or a product of an OR related to a probability of developing the disease and a PAF.

6. The method of claim 1, wherein the consideration of weights in proportion to the influence on disease occurrence with respect to the second value comprises clustering one or more monogenic variants into one or more clusters, wherein the clustering utilizes any one unsupervised learning technique selected from the group consisting of hierarchical clustering, k-means clustering, mixture model clustering, density-based spatial clustering of applications with noise (DBSCAN), generative adversarial networks (GAN), and self-organizing map (SOM).

7. The method of claim 1, further comprising classifying the subject into a non-risk group, a risk group, a high-risk group, or a very high-risk group according to the disease occurrence risk.

8. The method of claim 1, wherein the sample from the subject is blood.

9. A computer-readable recording medium having recorded thereon a program for executing the method of claim 1, on a computer.

10. A computing device comprising:
at least one memory; and
at least one processor,
wherein the processor is configured to: select a single nucleotide polymorphism (SNP) associated with disease occurrence and a monogenic variant associated with disease occurrence; analyze, using a sample from a subject, the subject's information on the selected SNP and the subject's information on the selected monogenic variant; obtain a first value, which is a polygenic risk score (PRS) value for the disease obtained based on the selected SNP information, and a second value, which is a monogenic risk score (MRS) value for the disease obtained based on the selected monogenic variant information; and obtain an integrated genetic risk for the disease based on the obtained first and second values, thereby predicting the risk of the disease based on the genetic risk of the disease.

11. The computing device of claim 10, wherein an integrated genetic risk for the disease is calculated based on the obtained first and second values.

12. The computing device of claim 10, wherein the processor is configured to determine the first value based on the presence or absence of a SNP variant whose disease association corresponds to a top 10th percentile or that occurs with a frequency at least twice that observed in a control group.

13. The computing device of claim 10, wherein the processor is configured to determine the second value based on the presence or absence of a genetic variation in the monogenic variant selected as being associated with disease occurrence.

14. The computing device of claim 10, wherein the processor is configured to select the selected monogenic variant associated with disease occurrence by using an odds ratio (OR) related to a probability of developing the disease, a population attributable fraction (PAF), or a product of an OR related to a probability of developing the disease and a PAF.

15. The computing device of claim 10, wherein the processor is configured to classify the subject into a non-risk group, a risk group, a high-risk group, or a very high-risk group according to the disease occurrence risk.
